(19) United States
(12) Patent Application Publication
(10) Pub. No.: US 2025/0255485 A1
(43) Pub. Date: Aug. 14, 2025

(54) System and Method for Remotely Monitoring Pathological Functions and Conditions with Multi-Spectral, Chip-Based Lidar

(71) Applicant: SVI Technologies, Inc., Calabasas, CA (US)

(72) Inventor: M. Gregory Steinthal, Los Angeles, CA (US)

(73) Assignee: SVI Technologies, Inc., Calabasas, CA (US)

(21) Appl. No.: 19/049,496

(22) Filed: Feb. 10, 2025

Related U.S. Application Data
(60) Provisional application No. 63/552,140, filed on Feb. 10, 2024.

(51) Int. Cl. A61B 5/00

(52) U.S. Cl. CPC A61B 5/0077; A61B 5/0002; A61B 5/0064; A61B 5/0082; A61B 5/443; A61B 5/444; A61B 5/6801; A61B 5/7264; A61B 2560/0431; A61B 2560/0462

(57) ABSTRACT

Various implementations of the invention relate to a silicon-embedded multi-spectral lidar system and method for non-invasive, remote monitoring of pathological conditions of an individual. The system integrates multiple laser sources, photodetectors, and a processing module onto a silicon chip, enabling the capture and analysis of spatial, spectral, scattering and temporal data from a target subject (e.g., individual). By utilizing lidar signals at various wavelengths, various implementations of the invention detect three-dimensional (“3D”) surface and subsurface features such as micro-movements, vascular patterns, pigmentation changes, and tissue differences and/or irregularities. Machine learning algorithms process this information from the lidar to identify and classify abnormalities, enabling applications such as heart rate and respiration monitoring, eye movement tracking for neurological and mental health assessment, and detection of pathological skin conditions like cancer. Various implementations of the invention operate in real-time, adapt to environmental conditions, and support dynamic monitoring over time, thereby providing a comprehensive, non-contact solution for diagnostics and telemedicine, for example. Various implementations of the invention may comprise a compact silicon chip form factor suitable for portable and/or hand-held, scalable health monitoring applications across diverse environments.

### CROSS-REFERENCE TO RELATED APPLICATIONS

This Application claims priority to U.S. Provisional Patent Application No. 63/552, 140, filed on Feb. 10, 2024, which is incorporated herein by reference in its entirety.

### FIELD OF THE INVENTION

The invention is generally related to monitoring physiological or pathological functions and/or conditions of an individual, and more specifically to remotely monitoring such physiological or pathological functions and/or conditions with a multi-spectral, chip-based lidar.

### BACKGROUND OF THE INVENTION

Various technologies exist for monitoring the physiological functions of a subject, including those related to cardiovascular and respiratory systems, as well as other bodily functions. Cardiovascular parameters may encompass heart rate, heart rate variability, pulse transit time, pulse shape, and other related metrics. Respiratory functions can include respiration rate, respiratory effort, and additional respiratory measures.

Other conventional monitoring devices generally require close physical proximity or direct access to the subject to measure these physiological functions. For instance, cardiovascular functions may be tracked through direct contact (such as observing blood flow through surface blood vessels or detecting heartbeats via chest movement) or via sound-based methods (e.g., using a stethoscope), both of which require physical proximity. Respiratory functions, on the other hand, may be monitored by measuring the flow of air near the subject's airway or employing other techniques that necessitate close interaction.

However, the need for physical contact or proximity with some conventional devices can be intrusive and uncomfortable for the subject, which may limit their use in certain situations. In some cases, this can prevent discreet monitoring, or it may interfere with the subject's ability to rest or go about their normal activities during monitoring. These limitations, among others, are common drawbacks of conventional monitoring methods.

Advancements in technology have enabled the monitoring of neurological and mental health functions by tracking eye movements and related ocular parameters. Eye movement patterns can provide valuable insights into cognitive functions, mental states, and neurological conditions. These include tracking parameters such as gaze direction, pupil dilation, blink rate, and saccadic movements, which can be indicative of attention, stress levels, cognitive load, and potential neurological disorders.

Conventional methods of assessing neurological and mental health typically involve physical assessments, clinical evaluations, or use of specialized equipment that requires direct interaction with the subject. For example, eye movements may be monitored by tracking devices that require the subject to wear sensors or undergo specific eye tracking procedures. These assessments often require the subject to be in close proximity to the equipment or a clinician, which may limit the frequency or ease of monitoring.

The reliance on physical proximity or direct interaction with the subject can be intrusive or uncomfortable, making continuous or discreet monitoring challenging. In many cases, such devices may interfere with the subject's natural behavior or mental state, affecting the accuracy of the data collected. These limitations can also prevent the monitoring of subjects in everyday environments or during spontaneous activities, hindering the ability to gather natural, real-time data. Other challenges and constraints associated with traditional eye tracking technologies are also recognized in the field.

Recent advancements in medical technology have enabled the detection of certain pathological conditions, such as, but not limited to, skin cancer, through non-invasive monitoring techniques. This medical technology can analyze various skin parameters, such as color, texture, and irregularities in skin lesions, etc., to identify potential signs of skin cancer, including melanoma and other skin abnormalities. Technologies such as optical imaging, multispectral imaging, and hyperspectral imaging are commonly used to detect early-stage skin cancer by analyzing changes in skin appearance that may indicate abnormal cell growth or malignancy.

Conventional technologies for skin cancer detection typically involve physical examinations by healthcare professionals or diagnostic procedures like biopsy, which may require direct contact and often invasive techniques. These technologies can be uncomfortable for the patient and may require significant time and resources. In addition, regular physical check-ups may be necessary to detect early signs of skin cancer, especially in patients with high-risk factors.

The need for physical examinations or invasive procedures often limits the frequency and accessibility of skin cancer monitoring, making early detection more challenging. Moreover, these conventional methods may not be suitable for continuous or remote monitoring, which can hinder timely detection and intervention. These limitations, combined with the discomfort and potential risks associated with invasive methods, highlight the challenges in effectively monitoring skin health and detecting skin cancer at its earliest stages.

### SUMMARY OF THE INVENTION

According to various implementations of the invention, a lidar for monitoring conditions of a target includes a silicon chip, where the silicon chip includes: a plurality of laser sources, each of the plurality of laser sources configured to emit signals at a different wavelength, an optical signal processor configured to receive signals emitted from the plurality of laser sources that are incident on and reflected and scattered from the target, to differentiate the received signals based on wavelength, and to generate three-dimensional spatial data, spectral reflectance data, optical scattering data, temporal data, surface and subsurface depth profiling of the target based on the differentiated received signals, and a machine learning engine configured to identify conditions of the target from the three-dimensional spatial data, spectral reflectance data, optical scattering data, temporal data, and surface and subsurface depth profiling of the target; and a data interface configured to transmit the identified conditions to a monitoring system or display.

In some implementations of the lidar, the machine learning engine is further configured to: differentiate spectral signatures of skin, vascular networks, and tissues from the multi-dimensional spatial data, the spectral reflectance data, optical scattering data, temporal data, surface and subsurface depth profiling of the target; and identify pathological changes in skin, such as pigmentation and structural irregularities, indicative of cancerous or precancerous conditions.

In some implementations of the lidar, the different wavelengths of the laser sources are configured to detect different vascular patterns, different subsurface tissue structures, or different pigmentations.

In some implementations of the lidar, the different wavelengths of the laser sources are configured to penetrate clothing or eyeglass lenses.

In some implementations of the lidar, the plurality of laser sources comprises a low power, tunable laser diode array.

In some implementations of the lidar, the lidar is portable.

In some implementations of the lidar, the lidar is handheld.

In some implementations of the lidar, the lidar is wearable.

In some implementations of the lidar, the machine learning engine is further configured to identify and differentiate between different types of skin lesions, including benign skin lesions and malignant skin lesions.

In some implementations of the lidar, the optical signal processor is further configured to filter environmental interferences or other noise.

In some implementations of the lidar, the wavelengths of one or more of the plurality of laser sources are adjustable.

In some implementations of the lidar, the wavelengths of one or more of the plurality of laser sources is adjustable based on a skin type of the target.

In some implementations of the lidar, the wavelengths of one or more of the plurality of laser sources includes an infrared wavelength configured to penetrate tissue or detect vascular anomalies at different depths.

In some implementations of the lidar, the wavelengths of one or more of the plurality of laser sources are adjustable based on environmental conditions in which the lidar is operating or in which the target resides.

In some implementations of the lidar, the lidar monitors conditions of a plurality of targets.

In some implementations of the lidar, the lidar is integrated with a telemedicine platform configured to remotely diagnose the pathological changes identified by the machine learning engine.

According to various implementations of the invention, a method for monitoring conditions of a target includes: emitting, from a lidar and toward the target, a plurality of signals, each of the plurality of signals having a different wavelength; receiving, by the lidar, signals that are incident on and reflected and scattered from the target; differentiating the received signals based on their wavelength; generating spatial data, spectral data, temporal data, optical scattering data, and surface and sub-surface depth profiling from the differentiated received signals; identifying, by a machine learning engine, conditions of the target from the spatial data, spectral data, and temporal data; and transmitting the conditions of the target to a display or a remote monitoring system.

### BRIEF DESCRIPTION OF THE DRAWINGS

FIG. 1 is a block diagram of a multi-spectral lidar on a silicon chip according to various implementations of the invention.

FIG. 2 is an image rendering of a multi-spectral lidar on a silicon chip according to various implementations of the invention.

FIG. 3 illustrates various monitoring of a target (e.g., individual) according to various implementations of the invention.

FIG. 4 illustrates a tissue cross-section being sampled by a conventional lidar.

FIGS. 5A and 5B illustrate a tissue cross-section being sampled by a lidar according to various implementations of the invention.

### DETAILED DESCRIPTION OF THE INVENTION

Various implementations of the invention relate to remotely monitoring an individual, more particularly, remotely monitoring one or more physiological functions, neurological, mental health and pathological functions and and/or conditions of the individual using a multi-spectral lidar. Multi-spectral lidar offers significant advantages over single-wavelength systems for monitoring physiological functions and conditions by providing greater accuracy, depth penetration, and adaptability. By utilizing multiple wavelengths, including wavelengths in different spectral regions, multi-spectral lidar enhances tissue and function differentiation, allowing for more precise measurement of physiological processes such as oxygenated blood flow, neural activity, and muscle contractions. Multi-spectral lidar's ability to capture multi-layer depth information enables comprehensive imaging of both surface and deeper biological structures, making it particularly valuable for monitoring neurological and cardiac functions. Additionally, multi-spectral lidar ensures higher functional accuracy and sensitivity by cross-validating signals from different wavelengths, reducing errors and improving measurement reliability. This adaptability extends to improved real-time sensing as seen over conventional, single-frequency lidar, where different wavelengths can be optimized dynamically to track changes in physiological conditions, such as stress or fatigue. Furthermore, by filtering out background noise and isolating relevant signals, multi-spectral lidar significantly improves signal integrity, making it more resilient to environmental interference.

Multi-spectral lidar offers a variety of implementations for remote heart rate detection across various applications. In some implementations, the lidar may monitor skin surface motion, which measures and/or detects subtle chest or skin movements caused by the heartbeat. Some implementations utilize near-infrared wavelengths (e.g., 1550 nm) to penetrate thin clothing for health monitoring.

In some implementations, the lidar may monitor blood flow and pulse detection, capturing micro-vibrations in arteries, such as a carotid artery, by optimizing specific near-infrared bands. As an example, a multi-spectral lidar system using near-infrared (NIR) wavelengths may significantly enhance blood flow monitoring, pulse detection, and arterial micro-vibration analysis by leveraging key wavelengths optimized for different physiological functions. Wavelengths of 760 nm and 850 nm enable blood oxygenation monitoring by distinguishing oxygenated and deoxygenated hemoglobin, while wavelength 940 nm penetrates deeper for arterial pulse wave analysis. Wavelength 1064 nm reduces scattering, improving arterial wall motion tracking, and wavelength 1310 nm enhances vascular imaging and pulse wave velocity measurements. Finally, wavelength 1550 nm, an eye-safe wavelength, is ideal for high-resolution Doppler-based arterial micro-vibration detection. Together, these NIR wavelengths provide multi-depth, high-sensitivity monitoring, making them applicable for non-contact cardiovascular assessments.

In such implementations, monitoring blood flow and pulse detection may aid in assessing stress levels of the individual, in security systems, for example. In some implementations, the lidar may provide subsurface depth profiling which combines visible and infrared wavelengths to separate surface-level chest movements from subsurface blood vessel dynamics, enabling advanced diagnostics for healthcare or fitness monitoring.

In some implementations, the lidar may compensate for environmental challenges through ambient condition compensation, where multiple spectral bands may be adjusted, in some cases, automatically, to adapt to noise from lighting, motion, or vibrations, making it ideal for outdoor monitoring of individuals, such as athletes or workers.

In some implementations, the lidar may monitory multi-parameter vital signs, where a combination of visible, near-infrared, and thermal wavelengths simultaneously tracks heart rate, respiration rate, and skin temperature, providing comprehensive health assessments. Again, some implementations may utilize near-infrared wavelengths to penetrate thin clothing for health monitoring, allowing discreet monitoring in public security or sensitive environments.

In some implementations, the lidar may assist with behavioral analysis, for example, by monitoring and/or detecting stress heart rate variability (HRV) using infrared wavelengths to infer emotional states, making it valuable in security or surveillance. In some implementations, the lidar may assist with remote fitness tracking using visible wavelengths for reflective skin signals and near-infrared wavelengths for blood flow detection. Such implementations offer real-time, non-invasive athlete monitoring.

In some implementations, the lidar may provide long-range monitoring for defense and military settings. Such implementations may employ higher-power lasers at various wavelengths to detect heart rate at significant distances, for example, such as, but not limited to 10-20 meters, or more, to remotely monitor and detect soldiers' vitals. In some implementations, the lidar may isolate, detect, and monitor heart rates of multiple individuals using different spectral bands, providing crowd monitoring capabilities for health and security applications. Such implementations highlight the versatility of various implementations of the invention in addressing diverse challenges and needs in remote heart rate detection. In some implementations of the invention, the lidar may be integrated into a security system to monitor stress and/or other anomalies associated with malintent.

Various implementations of the invention may track eye movements to assess neurological functions and mental health conditions. In some implementations, the lidar may precisely track eye movements using near-infrared wavelengths to detect subtle movements, such as saccades, gaze instability, and blink patterns, which may be indicative of conditions like Parkinson's or ALS. In some implementations, the lidar may monitor and/or detect gaze by combining visible wavelengths for pupil tracking with infrared wavelengths for eyelid motion detection, enabling assessments of attention, emotional states, and/or conditions like ADHD or anxiety. In some implementations, the lidar may monitor blink rate and fatigue by leveraging multi-spectral bands to detect changes in blink frequency and duration, which may signal stress or cognitive overload. In some implementations, the lidar may provide depth profiling using multiple wavelengths to distinguish surface-level eye movements from underlying muscle activity around the eyes, improving diagnostic accuracy. In some implementations, the lidar may utilize multiple spectral bands to compensate for variable lighting conditions or obstructions like glasses, ensuring robust monitoring across environments. In some implementations, the lidar may be used for REM sleep monitoring, tracking rapid eye movements during sleep to evaluate sleep quality and its relationship to mental health.

In some implementations, the lidar may monitor multiple eye parameters including, but not limited to eye movement, pupil dilation, and skin vibration data for a comprehensive understanding of cognitive load or emotional states. Such implementations may be used to monitor groups of individuals (e.g., crowds, audiences, etc.) using multi-spectral data to enable simultaneous tracking of multiple individuals, making it useful for stress detection in surveillance or security applications.

In some implementations, the lidar may utilize both visible and near infrared wavelengths to analyze pupil and iris behavior, which may be indicative of neurological impairments. In some implementations, the lidar may utilize infrared wavelengths to track eye movements through transparent barriers like glasses, ensuring usability in real-world scenarios.

In some implementations, the lidar may detect various pathological conditions, including, for example, skin cancer. Such implementations leverage the multi-spectral lidar's precision, depth profiling, and spectral analysis capabilities. In some implementations, the lidar may measure and creating subsurface high-resolution three-dimensional (“3D”) maps of skin lesions. Such maps may capture surface texture, elevation, and depth irregularities or lesions that may be indicative of malignancy.

In some implementations, the lidar may distinguish between different types of tissue depending upon various reflectance, absorption, and scattering responses of such tissue to different wavelengths, thereby differentiating between tissue types (e.g., skin, normal fat or muscle tissue, abnormal tissue, scar tissue, etc). In some implementations, the lidar may detect subsurface features, such as vascular patterns or pigmentation abnormalities by penetrating shallow layers of the skin using near-infrared wavelengths. In some implementations, the lidar may monitor changes in such tissue over time to track lesion changes in size, color, or depth, etc., to support early detection and disease progression analysis.

In various implementations of the invention, the lidar offers advantages over other systems by providing deeper, more detailed, and multi-dimensional insights into biological processes, ensuring higher accuracy, sensitivity, and adaptability across a wide range of conditions. For example, detecting melanoma through conventional multispectral imaging techniques, typical wavelengths of light are chosen to target specific chromophores. A typical multispectral imaging device uses 5-10 distinct wavelengths as the different wavelengths penetrate the skin to varying depths, allowing simultaneous examination of surface features (epidermis) and subsurface structures (dermis and vascular layers). This multi-depth information provided by the multiple spectra reduces the risk of missing key diagnostic features associated with melanoma by examining potential lesions across multiple spectral bands because melanoma can affect both the surface (e.g., pigmentation) and deeper structures (e.g., blood vessels). Further, melanoma lesions that appears benign at one wavelength may exhibit malignant characteristics at another wavelength; for example, a pigmented lesion with normal surface features might have irregular blood flow underneath, indicative of melanoma.

Although conventional multispectral imaging systems capture reflected light at different wavelengths, they do not directly measure depth. Depth penetration in such conventional systems is inferred indirectly by analyzing wavelength-specific reflectance or absorption, thereby providing only qualitative or semi-quantitative information about various tissue layers.

In contrast, the lidar in accordance with various implementations of the invention provides three-dimensional (“3D”) subsurface spectroscopic depth profiling, enabling real-time, detailed mapping of tissue composition at various depths, as well as the precise geometry of subsurface structures. Unlike conventional multispectral imaging, which focuses on reflectance and pigmentation analysis, such lidar introduces depth resolution, active illumination, and advanced scattering analysis. By detecting true depth-related changes in tissue composition and identifying abnormal growth beneath the skin-capabilities beyond standard imaging-such creates detailed 3D profiles of skin lesions, including subsurface structures, thereby offering a more comprehensive analysis of skin lesions, enabling earlier detection and significantly improving diagnostic accuracy.

Additionally, by analyzing the multispectral data from the lidar with machine learning, automated classification of benign and malignant lesions becomes feasible identifying patterns and classifying lesion with high accuracy while reducing reliance on biopsies.

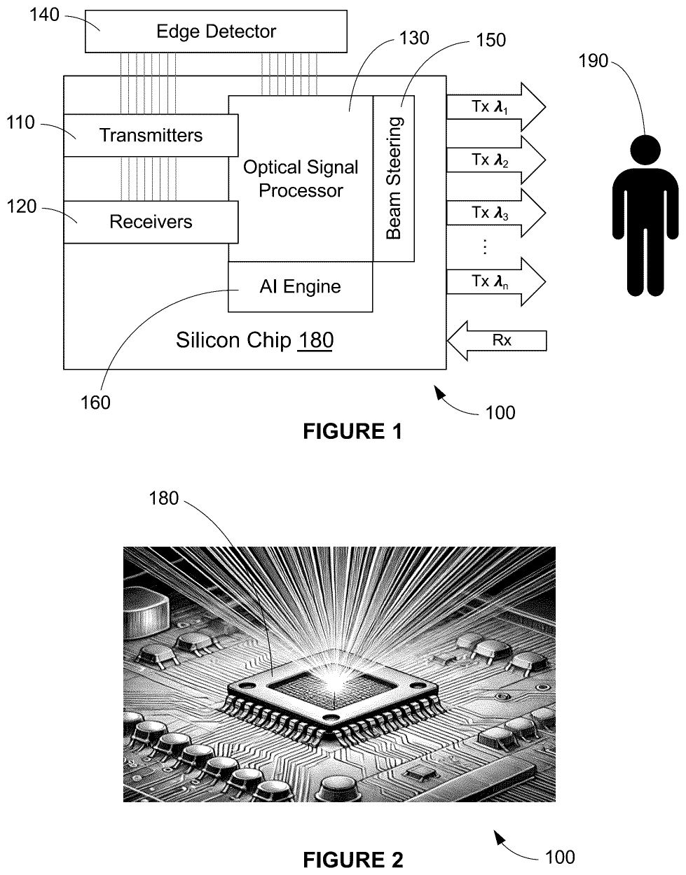

FIG. 1 is an exemplary block diagram of a multi-spectral lidar 100 for remotely monitoring an individual 190, in accordance with various implementations of the invention. In some implementations of the invention, lidar 100 is a frequency modulated, continuous wave (“FMCW”) multi-spectral lidar. In some implementations, lidar 100 includes a number of discrete laser sources (illustrated in FIG. 1 as transmitters 110), each tuned to a particular frequency, or finite range of frequencies, as would be apparent. In some implementations of the invention, lidar 100 includes a tunable laser source (replacing transmitters 110 in FIG. 1 with a signal transmitter 110) tunable to a plurality of lidar frequencies as would be apparent. Transmitters cause various signals to be emitted from lidar 100 (illustrated in FIG. 1 as emitted signal Tx λ₁, emitted signal Tx λ₂, emitted signal Tx λ₃, . . . emitted signal Tx λₙ), each emitted at a different discrete wavelength, λᵢ. Lidar 100 also includes of one or more photo detectors (illustrated in FIG. 1 as receivers 120) that detect the emitted signal(s) Tx λᵢ that are incident on and reflected from of a distance object, such as individual 190. Lidar 100 includes an optical signal processor 130 configured to perform wavelength division multiplexing to allow for simultaneous collection of data at the various wavelengths of the emitted signals as would be appreciated. The wavelength division multiplexing of optical signal processor 130 enhances target differentiation and improves resolution of various aspects of individual 190. Lidar 100 also includes an edge detector 140 configured to process light reflected from individual 190 based on reflectance, scattering and/or polarization properties of various aspects of individual 190. In some implementations of the invention, lidar 100 includes a beam steering module 150, such as an optical phase array or other beam steering device, configured to provide active beam steering of the emitted signals as would be appreciated.

In some implementations of the invention, lidar 100 is configured on a silicon chip 180 as would be appreciated. In some implementations of the invention, multiple single wave-length lidar chips, each with a different wavelength, may be cascaded to form a multispectral system as would be appreciated. In some implementations of the invention, lidar 100 includes an AI engine 160. AI engine 160 enhances real-time signal processing by filtering out noise, motion artifacts, and unwanted reflections, ensuring high-quality data. AI engine 160 also provides AI-driven multi-wavelength data fusion to intelligently combine signals to improve contrast, optimize lidar parameters, and adapt to changes. By leveraging pattern recognition, AI engine 160 may, for example, detect irregular blood flow, arterial stiffness, and early signs of cardiovascular issues, enabling predictive diagnostics. Additionally, automated feature extraction allows AI engine 160 to analyze pulse wave velocity and micro-vibration signatures and more providing decision prediction output capabilities.

AI engine 160 is configured to receive information from optical signal processor 130 and/or edge detector 140 regarding individual 190. Such information may include three-dimensional spatial data, spectral reflectance data, and/or temporal data, all associated with various aspects of individual 190. Utilizing various machine learning techniques, AI engine 160 is configured to distill such information into various diagnostic information regarding individual 190 as would be appreciated. In some implementations of the invention, AI engine 160 may be configured with lidar 100 on silicon chip 180 (as illustrated). In some implementations of the invention, AI engine 160 may be configured off-chip as would be appreciated. In some implementations of the invention, AI engine 160 may be configured remote from lidar 100, such as in the cloud or other back-end server. In some implementation of the invention, AI engine 160 may be fused with other sensing modalities beyond lidar 100 to further increase accuracy as would be appreciated.

FIG. 2 is an image rendering 200 of lidar 100 on silicon chip 180 according to various implementations of the invention.

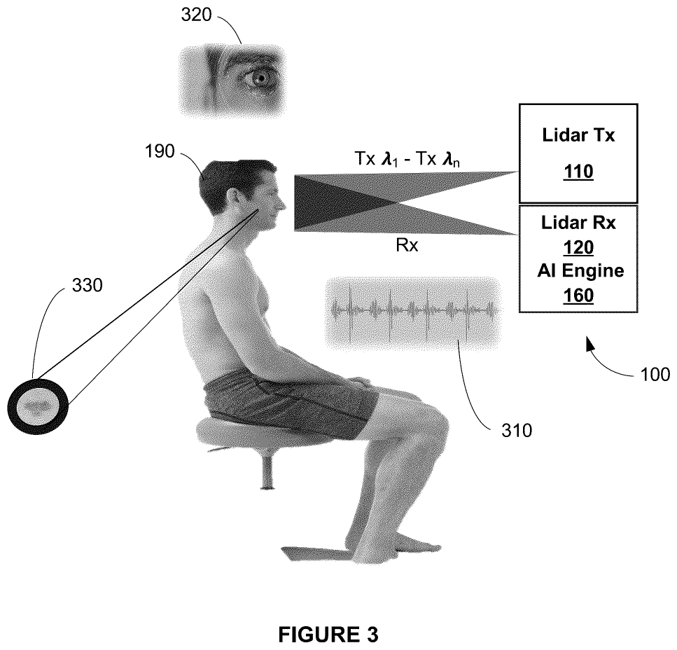

FIG. 3 illustrates various monitoring of a target (e.g., individual 190) according to various implementations of the invention. In some implementations, lidar 100 transmits or emits a plurality of signals Tx A; toward individual 190 to, for example, perform a facial scan (i.e., emitted signals incident on and reflected back from a face of individual 190). In this example, the facial scan may include various aspects of individual 190, including, for example, but not limited to, skin vibrations caused by including blood flow in the forehead and/or cheek area, heart beat rates, breathing rates, pulse, etc. Such information may be extracted by optical signal processor 130 alone or in combination with AI engine 160. Information extracted from skin vibrations may be further evaluated by AI engine 160 to determine various respiratory and/or cardiac information 310, including, for example, but not limited to: rhythm abnormalities used to monitor exercise response, resting heart rate, heart rate variability (HRV), diagnose tachycardia and bradycardia conditions, responses to medications or interventions, and other such information as would be appreciated.

In another example (or in addition to the example above), the facial scan may detect and track various aspects of individual 190, including eye movement and tracking information 320 including, for example, but not limited to: various eye movements, including eye velocity, eye acceleration, eye position, or other eye movement information associated with eye(s) of individual 190. Again, such information may be extracted by optical signal processor 130 alone or in combination with AI engine 160. Information extracted from eye movements may be further evaluated by AI engine 160 to determine (or assess), for example, but not limited to: oculomotor functions, visual perception, attention disorders, executive functions, spatial awareness, vestibular functions, early signs of Parkinson's disease and other eye-related assessment. Further, certain mental health conditions may also be diagnosed from eye movements and tracking information 320, such as, but not limited to: Attention Deficit Hyperactivity Disorder (ADHD), Anxiety Disorders, Depression, Post-Traumatic Stress Disorder (PTSD), Schizophrenia Spectrum Disorders, Autism Spectrum Disorder (ASD), Bipolar Disorder, etc.

In some implementations of the invention, respiratory and/or cardiac information 310 and eye movements and tracking information 320 may be monitored together, for example, to analyze sleep quality as would be appreciated, among other things. (57) In another example (or in addition to the example(s) above), the facial scan may penetrate various surfaces of individual 190 to detect and measure various aspects of individual 190, including various spectroscopic information 330 of individual 190, including, for example, various skin and/or tissue skin information or conditions with little, if any, physical intrusion.

The multi-spectral transmit signals of lidar 100 are configured to penetrate skin and/or tissue of individual 190 at different skin depths resulting in scans that will produce a detailed, multi-layered image of the skin. The spectroscopic data 330 may provide insights into the cellular and subcellular structures of the skin as the presence of cancerous cells may alter the light scattering properties of skin due to the differences in physical and biological properties of cancerous cells compared to normal cells. According to various implementations of the invention, AI engine 160 may be configured to identify slight variations in skin density or irregularities in cell formation, which may enable early detection of various skin or tissue anomalies, including melanoma or other skin cancers. Additionally, AI engine 160 may be configured to detect changes in pigmentation beneath the surface layer of skin, which might not yet be visible externally, revealing minute changes in skin structure, which are often imperceptible to the naked eye, and allowing for early intervention.

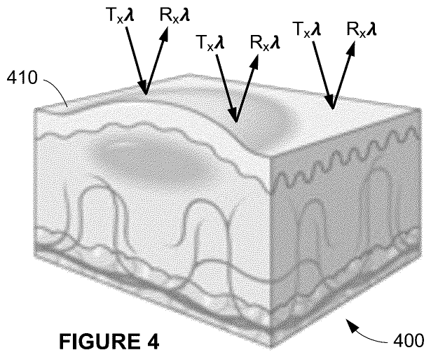

FIG. 4 illustrates a tissue cross-section 400 on which transmit signals (illustrated in FIG. 4 as transmit signals Tₓλ) from a conventional lidar are incident on and reflected off a surface 410 and received by the conventional lidar as receive signals (illustrated in FIG. 4 as receive signals Rₓλ). Transmit signals from conventional lidar reflect from surface 410 and may be used to obtain accurate depictions of surface 410 of tissue cross-section 400. However, because conventional lidar typically employs a single wavelength laser source, transmit signals typically do not penetrate tissue cross-section much beyond surface 410 and hence, cannot depict structures below surface 410.

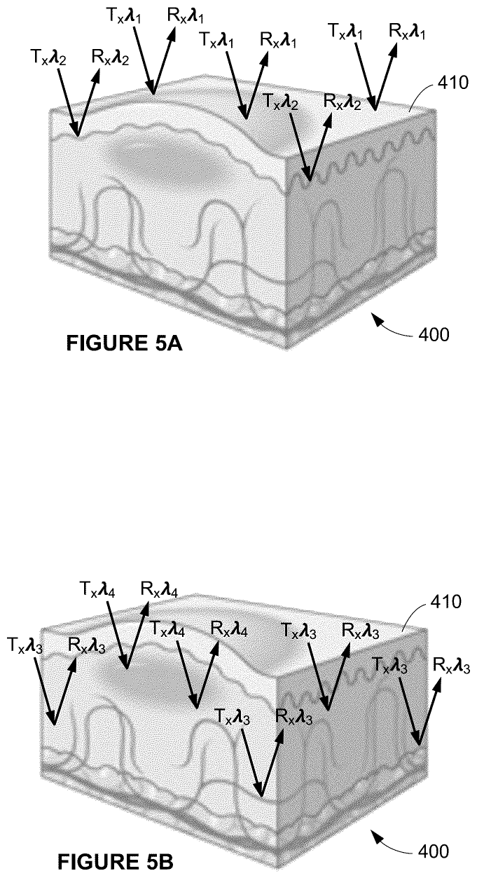

FIGS. 5A and 5B illustrate tissue cross-section 400 on which transmit signals from lidar 100 are incident on surface 410. FIG. 5A illustrates that certain transmit signals from lidar 100 are incident on tissue cross-section 400 and interact with it through reflection, absorption, scattering, etc. (illustrated in FIG. 5A as transmit signals Tₓλ₁ receive signals Rₓλ₁ interacting with surface 410) and other transmit signals from lidar 100 penetrate surface 410 into tissue cross-section 400 and interact with various sub-surface structures within tissue cross-section 400 (illustrated in FIG. 5A as transmit signals Tₓλ₂ and receive signals Rₓλ₂). This is accomplished by selecting different wavelengths (which may include wavelengths of different spectra) for transmit signals (i.e., λ₁ vs. λ₂). For ease of illustration, FIG. 5B likewise illustrates additional transmit signals and their corresponding receive signals (illustrated in FIG. 5B as transmit and receive signals Tₓλ₃, Rₓλ₃ and transmit and receive signal Tₓλ₄, Rₓλ₄) that may penetrate differently or further into cross-section 400 and reflect of other sub-surface structures as would be appreciated. While FIGS. 5A and 5B collectively illustrate four different wavelengths of transmit signals, lidar 100 may include fewer or more wavelengths depending upon application, nature of tissue, etc., as would be appreciated. The multi-wavelength and/or multi-spectral transmit signals facilitate a detailed, multi-layered depth profile of tissue cross section 400. While FIGS. 5A and 5B illustrate transmit signals incident on surfaces or sub-surface structures within tissue cross-section 410 and being “reflected” therefrom, reflectance is not the only information gleaned from the received signals; other or additional information including scattering, absorption, multi-path may be extracted via various optical signal processing techniques as would be appreciated. In various implementations of the invention, this depth profile may be displayed holographically, in augmented reality, on a three-dimensional display, or viewed through cross-sectional and layered rendering as would be appreciated.

According to various implementations of the invention, lidar 100 provides remote, multi-spectral, non-contact, non-invasive monitoring of respiratory and cardiac information 310 eye movement and tracking information 320, and spectroscopic information In some implementations of the invention, lidar 100 may be configured as a standalone system or configured a web-cam format for telemedicine. In some implementations of the invention, lidar 100 may be a handheld, or otherwise portable, device providing convenience of use, etc., as would be appreciated.

While the invention has been described herein in terms of various implementations, it is not so limited and is limited only by the scope of the following claims, as would be apparent to one skilled in the art.

What is claimed is:

1. A lidar for monitoring conditions of a target comprising:
a silicon chip, the silicon chip comprising:
a plurality of laser sources, each of the plurality of laser sources configured to emit signals at a different wavelength,
an optical signal processor configured to receive signals emitted from the plurality of laser sources that are incident on and reflected and scattered from the target, to differentiate the received signals based on wavelength, and to generate three-dimensional spatial data, spectral reflectance data, optical scattering data, temporal data, surface and subsurface depth profiling of the target based on the differentiated received signals, and
a machine learning engine configured to identify conditions of the target from the three-dimensional spatial data, spectral reflectance data, optical scattering data, temporal data, and surface and subsurface depth profiling of the target; and
a data interface configured to transmit the identified conditions to a monitoring system or display.

2. The lidar of claim 1, wherein the machine learning engine is further configured to:
differentiate spectral signatures of skin, vascular networks, and tissues from the multi-dimensional spatial data, the spectral reflectance data, optical scattering data, temporal data, and surface and subsurface depth profiling of the target; and
identify pathological changes in skin, such as pigmentation and structural irregularities, indicative of cancerous or precancerous conditions.

3. The lidar of claim 1, wherein the different wavelengths of the laser sources are configured to detect different vascular patterns, different subsurface tissue structures, or different pigmentations.

4. The lidar of claim 1, wherein the different wavelengths of the laser sources are configured to penetrate clothing or eyeglass lenses.

5. The lidar of claim 1, wherein the plurality of laser sources comprises a low power, tunable laser diode array.

6. The lidar of claim 5, wherein the lidar is portable.

7. The lidar of claim 5, wherein the lidar is handheld.

8. The lidar of claim 5, wherein the lidar is wearable.

9. The lidar of claim 2, wherein the machine learning engine is further configured to identify and differentiate between different types of skin lesions, including benign skin lesions and malignant skin lesions.

10. The lidar of claim 1, wherein the optical signal processor is further configured to filter environmental interferences or other noise.

11. The lidar of claim 1, wherein the wavelengths of one or more of the plurality of laser sources is adjustable.

12. The lidar of claim 11, wherein the wavelengths of one or more of the plurality of laser sources is adjustable based on a skin type of the target.

13. The lidar of claim 1, wherein the wavelengths of one or more of the plurality of laser sources includes an infrared wavelength configured to penetrate tissue or detect vascular anomalies at different depths.

14. The lidar of claim 11, wherein the wavelengths of one or more of the plurality of laser sources is adjustable based on environmental conditions in which the lidar is operating or in which the target resides.

15. The lidar of claim 1, wherein the lidar monitors conditions of a plurality of targets.

16. The lidar of claim 2, wherein the lidar is integrated with a telemedicine platform configured to remotely diagnose the pathological changes identified by the machine learning engine.

17. A method for monitoring conditions of a target comprising:
emitting, from a lidar and toward the target, a plurality of signals, each of the plurality of signals having a different wavelength;
receiving, by the lidar, signals that are incident on and reflected and scattered from the target;
differentiating the received signals based on their wavelength;
generating spatial data, spectral data, temporal data, optical scattering data, and surface and sub-surface depth profiling from the differentiated received signals;
identifying, by a machine learning engine, conditions of the target from the spatial data, spectral data, and temporal data; and
transmitting the conditions of the target to a display or a remote monitoring system.
